(19) United States
(12) Patent Application Publication
(10) Pub. No.: US 2023/0037938 A1
(43) Pub. Date: Feb. 9, 2023

(54) Vitamin Testing Diaper Device

(71) Applicant: Daniel Nicol

(72) Inventor: Daniel Nicol

(21) Appl. No.: 17/878,267

(22) Filed: Aug. 1, 2022

Related U.S. Application Data
(60) Provisional application No. 63/228,786, filed on Aug. 3, 2021.

(51) Int. Cl. A61F 13/84; A61B 10/00

(52) U.S. Cl. CPC A61F 13/84; A61B 10/007; A61B 2010/0006; A61F 2013/8473

(57) ABSTRACT

The present invention relates generally to the field of diapers. More specifically, the present invention relates to a vitamin testing diaper device primarily comprised of a body, further comprised of a pair of leg openings, at least one pad, and at least one testing strip. The testing strip is located on the pad. The testing strip is absorbent and impregnated with a chemical that reacts with urine such that the strip will change in color to indicate the presence of a substance in the urine. The substance may be, but is not limited to, a vitamin.

### CROSS-REFERENCE TO RELATED APPLICATION

The present application claims priority to, and the benefit of, U.S. Provisional Application No. 63/228,786 which was filed on Aug. 3, 2021, and is incorporated herein by reference in its entirety.

### FIELD OF THE INVENTION

The present invention relates generally to the field of diapers. More specifically, the present invention relates to a vitamin testing diaper device primarily comprised of a body, further comprised of a pair of leg openings, at least one pad, and at least one testing strip. The testing strip is located on the pad. The testing strip is absorbent and impregnated with a chemical that reacts with urine such that the strip will change in color to indicate the presence of a substance in the urine. The substance may be, but is not limited to, a vitamin. Accordingly, the present disclosure makes specific reference thereto. Nonetheless, it is to be appreciated that aspects of the present invention are also equally applicable to other like applications, devices, and methods of manufacture.

### BACKGROUND

Detecting vitamin deficiencies in infants and young children can be nearly impossible. Further, detecting vitamin deficiencies and other similar issues early on can prevent serious health issues, ultimately reducing healthcare costs for patients. In addition, future illnesses can lead to serious medical complications and reduced quality of life for an infant or child.

Therefore, there exists a long-felt need in the art for a convenient device that allows a parent or caregiver to easily detect a vitamin deficiency in an infant. In addition, there exists a long-felt need in the art for a vitamin testing diaper device that utilizes a diaper to easily detect a vitamin deficiency in an infant. Further, there exists a long-felt need in the art for a vitamin testing diaper device that utilizes a diaper to easily detect a vitamin deficiency in an infant, wherein the device is disposable.

The subject matter disclosed and claimed herein, in one embodiment thereof, comprises a vitamin testing diaper device. The device is primarily comprised of a body, further comprised of a pair of leg openings, at least one pad, and at least one testing strip. The testing strip is located on the pad and is absorbent and impregnated with a chemical. The chemical reacts with urine such that the strip will change in color to indicate the presence of a substance in the urine, wherein the substance may be but is not limited to a vitamin. The device is also comprised of a matrix card that allows a user to identify the concentration of the substance in the urine based on the specific color shade of the testing strip and by referencing the card.

In this manner, the vitamin testing diaper device of the present invention accomplishes all of the foregoing objectives and provides a convenient device that allows a parent or caregiver to easily detect a vitamin deficiency in an infant using a disposable device. In doing so, the device utilizes a diaper to easily detect a vitamin deficiency in an infant. This is further convenient since all infants use diapers.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a vitamin testing diaper device. The device is primarily comprised of a body, further comprised of a pair of leg openings, at least one pad, and at least one testing strip. The body of the device may be manufactured from a plurality of materials such as, but not limited to, wood pulp, sodium polyacrylate fibers, plastic (i.e., cellulose, polypropylene, polyethylene, etc.), tissue paper, non-woven fabric, non-permeable film, etc. In addition, the body and/or all or some of the components of the device may be infused with a plurality of substances unless otherwise stated, including, but not limited to, aloe vera, zinc oxide, calendula, lanolin, petroleum, and/or other moisturizing, anti-fungal, anti-bacterial compounds as are known in the art. The body has a pair of continuous leg openings that allow the legs of a user to be placed through the body while the device is worn. The body also has at least two tabs on the rear surface and front surface, which are each comprised of at least one fastener. The fastener allows the tabs to adhere to one another such that the body of the device can be secured around a user when worn.

At least one pad is located on the inner surface of the body. The top surface of the pad may further be infused with, or may be coated with, a lotion such as, but not limited to, aloe vera, zinc oxide, calendula, lanolin, petroleum, etc., and other suitable moisturizing, anti-fungal, or anti-bacterial compounds that prevent diaper rash, irritation, and/or infection. The pad is further absorbent.

The top surface of the pad is further comprised of at least one absorbent testing strip which is chemically impregnated with at least one chemical that is urine activated such that when urine saturates the testing strip, a chemical reaction occurs between the urine and the chemical that allows the strip to detect at least one substance in the urine and communicate the presence of said substance via a color change in the strip.

In one embodiment, the pad has a plurality of testing strips, wherein each strip may be colored coded to aid a parent or caregiver in identifying which testing strip detects which substance. The testing strip may also be labeled using at least one indicia that communicates the substance each strip detects. The testing strip preferably indicates the concentration of the substance in the urine via a unit such as, but not limited to: percentage, milligrams, IUs, micrograms, milliequivalents, etc.

The rear surface and front surface of the device area are also each comprised of at least two tabs that allow the device to be secured around a user's body. Once the tabs are secured, a pair of continuous leg openings allow the legs of a user to be placed through the body while the device is in use. Further, the tabs of the rear surface and front surface are each comprised of at least one fastener.

Accordingly, the vitamin testing diaper device of the present invention is particularly advantageous as it provides a convenient device that allows a parent or caregiver to easily detect a vitamin deficiency in an infant. The device utilizes a diaper to easily detect a vitamin deficiency in an infant. In this manner, the vitamin testing diaper device overcomes the limitations of existing diapers and vitamin testing methods known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a vitamin testing diaper device of the present invention in a semi-closed position in accordance with the disclosed architecture;

FIG. 2 illustrates a top view of one potential embodiment of a vitamin testing diaper device of the present invention in an opened position in accordance with the disclosed architecture; and

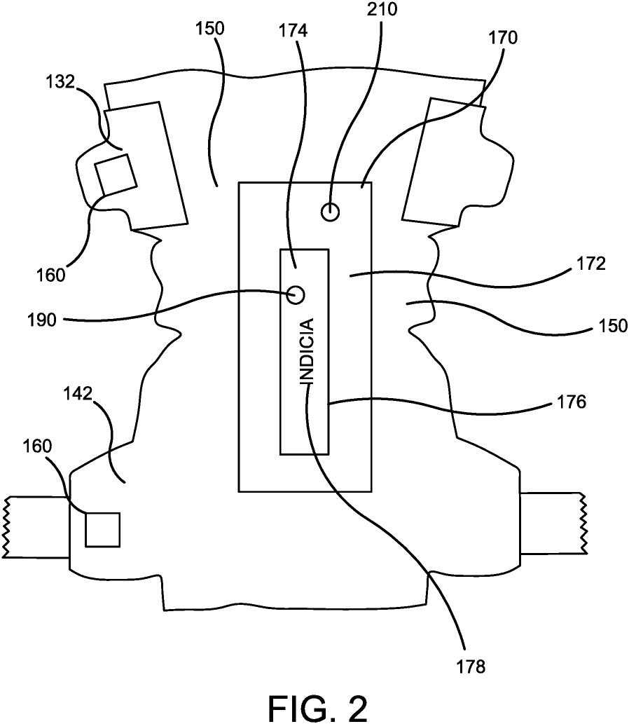

FIG. 3 illustrates a top view of one potential embodiment of a matrix card of one potential embodiment of a vitamin testing diaper device of the present invention in accordance with the disclosed architecture.

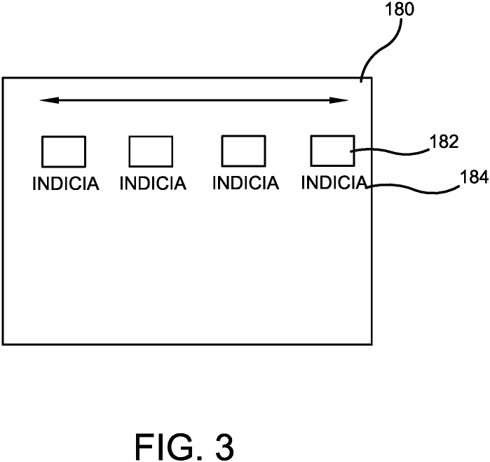

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that allows a parent or caregiver to easily detect a vitamin deficiency in an infant. In addition, there exists a long-felt need in the art for a vitamin testing diaper device that utilizes a diaper to easily detect a vitamin deficiency in an infant. Further, there exists a long-felt need in the art for a vitamin testing diaper device that utilizes a diaper to easily detect a vitamin deficiency in an infant, wherein the device is disposable.

The present invention, in one exemplary embodiment, is comprised of a vitamin testing diaper device. The device is primarily comprised of a body, further comprised of a pair of leg openings, at least one pad, and at least one testing strip. The body of the device may be manufactured of a plurality of materials such as, but not limited to, wood pulp, sodium polyacrylate fibers, plastic (i.e., cellulose, polypropylene, polyethylene, etc.), tissue paper, non-woven fabric, non-permeable film, etc. Further, the body and/or all or some of the components of the device may be infused with a plurality of substances unless otherwise stated, including, but not limited to, aloe vera, zinc oxide, calendula, lanolin, petroleum, and/or other moisturizing, anti-fungal, anti-bacterial compounds as are known in the art. The body has a pair of continuous leg openings that allow the legs of a user to be placed through the body while the device is worn. The body also has at least two tabs on the rear surface and front surface which are each comprised of at least one fastener. The fastener allows the tabs to adhere to one another such that the body of the device can be secured around a user when worn.

At least one pad is located on the inner surface of the body, wherein the top surface of the pad may further be infused with, or may be coated with, a lotion such as, but not limited to, aloe vera, zinc oxide, calendula, lanolin, petroleum, etc., and other suitable moisturizing, anti-fungal, or anti-bacterial compounds that prevent diaper rash, irritation, and/or infection. The pad is further absorbent.

The top surface of the pad is further comprised of at least one absorbent testing strip which is chemically impregnated with at least one chemical. The strip is urine activated such that when urine saturates the testing strip a chemical reaction occurs between the urine and the chemical that allows the strip to detect at least one substance in the urine and communicate the presence of said substance via a color change in the strip.

The pad may have a plurality of testing strips, wherein each strip may be colored coded to aid a parent or caregiver in identifying which testing strip detects which substance. The testing strip may also be labeled using at least one indicia that communicates the substance in which each strip detects. The testing strip preferably indicates the concentration of the substance in the urine via a unit such as, but not limited to: percentage, milligrams, IUs, micrograms, milliequivalents, etc.

The rear surface and front surface of the device are each also comprised of at least two tabs that allow the device to be secured the device around a user's body. Once the tabs are secured, a pair of continuous leg openings allow the legs of a user to be placed through the body while the device is in use. Further, the tabs of the rear surface and front surface are each comprised of at least one fastener.

Accordingly, the vitamin testing diaper device of the present invention is particularly advantageous as it provides a convenient device that allows a parent or caregiver to use a diaper to easily detect a vitamin deficiency in an infant. In this manner, the vitamin testing diaper device overcomes the limitations of existing diapers and vitamin testing methods known in the art.

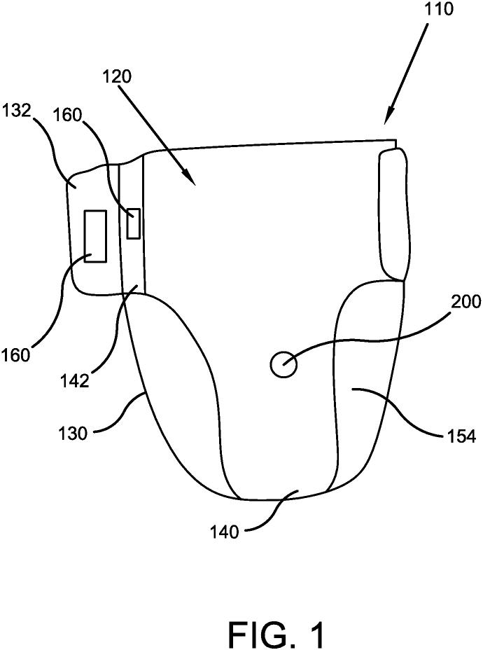

Referring initially to the drawings, FIG. 1 illustrates a perspective view of one potential embodiment of a vitamin testing diaper device 100 of the present invention in a semi-closed position in accordance with the disclosed architecture. The device is primarily comprised of a body 110, further comprised of a pair of leg openings 154, at least one pad 170, and at least one testing strip 174. The body 110 of the device 100 may be manufactured of a plurality of materials such as, but not limited to, wood pulp, sodium polyacrylate fibers, plastic (i.e., cellulose, polypropylene, polyethylene, etc.), tissue paper, non-woven fabric, non-permeable film, etc., or any other suitable diaper material as is known in the art or a combination thereof. In addition, the body 110 and/or all or some of the components of the device 100 may be infused with a plurality of substances 200 unless otherwise stated, including, but not limited to, aloe vera, zinc oxide, calendula, lanolin, petroleum, and/or other moisturizing, anti-fungal, anti-bacterial compounds as are known in the art. Further, the material of the body 110 may be hypoallergenic and/or scented or non-scented.

The body 110 has a pair of continuous leg openings 154 that allow the legs of a user to be placed through the body 110 while the device 100 is worn. The continuous leg openings 154 can be any suitable size as is known in the art and can be sized to fit an infant, a small child and/or an adult, depending on the needs and/or wants of a user. In one embodiment, the leg openings 152 may be elastic.

Additionally, the rear surface 130 of the device 100 comprises at least two tabs 132, and the front surface 140 of the device 100 comprises at least two tabs 142 to secure the device around a user's body. Once the tabs 132 and 142 are secured, a pair of continuous leg openings 154 allow the legs of a user to be placed through the body 110 while the device 100 is in use. Further, the tabs 132, 142 of the rear surface 130 and front surface 140 are each comprised of at least one fastener 160. Accordingly, the fastener 160 allows the tabs 132, 142 to adhere to one another, such that the body 110 of the device 100 can be secured around a user. In differing embodiments, the fastener 160 may be any suitable fastener as is known in the art, such as, but not limited to, a hook and loop and/or adhesive, etc.

At least one pad 170 is located on the inner surface 150 of the body 110. The pad 170 is preferably generally rectangular in shape but can be any suitable shape as is known in the art. Further, the pad 170 is typically manufactured of the same material as the body 110 but can be manufactured of any suitable material as is known in the art. The top surface 172 of the pad may further be infused with, or may be coated with, a lotion 210 such as, but not limited to, aloe vera, zinc oxide, calendula, lanolin, petroleum, etc., and other suitable moisturizing, anti-fungal, or anti-bacterial compounds that prevent diaper rash, irritation, and/or infection. The pad 170 is further absorbent.

The top surface 172 of the pad 170 is comprised of at least one absorbent testing strip 174. In one embodiment, the testing strip 174 is fixedly attached to the top surface 172 (i.e., part of the top surface 172). In another embodiment, the testing strip 174 has an adhesive rear surface 176 that allows the strip 174 to attach to the top surface 172. The testing strip 174 is chemically impregnated with at least one chemical 190 that is urine activated such that when urine saturates the testing strip 174, a chemical reaction occurs between the urine and the chemical 190 that allows the strip 174 to detect at least one substance in the urine and communicate the presence of said substance via a color change in the strip 174. In one embodiment, the strip 174 only detects the presence of a substance. In another embodiment, the strip detects the presence of and/or measures the quantity of a substance. Substances include, but are not limited to: Vitamin K, Vitamin D, Vitamin B12, Iron, Vitamin C, Vitamin A, Calcium, Vitamin E, Acidity (pH), protein, sugar, ketones, bilirubin, Leukocytes, red blood cells, etc.

In one embodiment, the pad 170 has a plurality of testing strips 174. Each strip 174 may be colored coded (i.e., wherein each strip 174 is a different color) to aid a parent or caregiver in identifying which testing strip 174 detects which substance. The testing strip 174 may also be labeled using at least one indicia 178 that communicates the substance each strip 174 detects in word, number, symbol, or image form. The testing strip 174 preferably indicates the concentration of the substance in the urine via a unit such as, but not limited to: percentage, milligrams, IUs, micrograms, milliequivalents, etc. To do so, the strip 174 has a spectrum of color shades that change in relation to the concentration of a substance in the urine. By way of example, a red-colored strip 174 for detecting Vitamin D will change color to a dark red color if a large amount of Vitamin D is detected and will change to a light red color if a small amount of Vitamin D is detected. The exact units that relate to each color shade can be obtained from at least one matrix card 180 that has at least one colored square 182. Each colored square 182 of the card 180 identifies each possible color shade of the strip 174 and what unit quantity of the substance each color shade represents. Each square 182 may have at least one indicia 184 that represents said unit quantity in word, number, symbol, or image form on the card 180.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein "vitamin testing diaper device" and "device" are interchangeable and refer to the vitamin testing diaper device 100 of the present invention.

Notwithstanding the foregoing, the vitamin testing diaper device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the vitamin testing diaper device 100 as shown in the FIGS. are for illustrative purposes only, and that many other sizes and shapes of the vitamin testing diaper device 100 are well within the scope of the present disclosure. Although the dimensions of the vitamin testing diaper device 100 are important design parameters for user convenience, the vitamin testing diaper device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term "includes" is used in either the detailed description or the claims, such term is intended to be inclusive in a manner similar to the term "comprising" as "comprising" is interpreted when employed as a transitional word in a claim.

What is claimed is:

1. A vitamin testing diaper device comprising:
a body;
a pair of leg openings in said body;
a front surface;
a tab and a fastener positioned on said front surface;
a rear surface;
a tab and a fastener positioned on said rear surface;
an inner surface;
a pad positioned on said inner surface; and
a testing strip.

2. The vitamin testing diaper device of claim 1, wherein a color of the testing strip changes when contacted with a urine substance.

3. The vitamin testing diaper device of claim 1, wherein the testing strip has an adhesive bottom surface that adheres to a top surface of the pad.

4. The vitamin testing diaper device of claim 2, wherein the testing strip is absorbent.

5. A vitamin testing diaper device comprising:
a body;
a pair of leg openings in said body;
a front surface;
a tab and a fastener positioned on said front surface;
a rear surface;
a tab and a fastener positioned on said rear surface;
an inner surface;
a pad positioned on said inner surface; and
a testing strip impregnated with a chemical.

6. The vitamin testing diaper device of claim 5, wherein the chemical reacts when exposed to urine.

7. The vitamin testing diaper device of claim 6, wherein the chemical causes a change in a color of the testing strip after the reaction occurs.

8. The vitamin testing diaper device of claim 7, wherein the change of the color of the testing strip indicates the presence of a substance.

9. The vitamin testing diaper device of claim 8, wherein the substance is a vitamin.

10. A vitamin testing diaper device comprising:
a body;
a pair of leg openings in said body;
a front surface;
a tab and a fastener positioned on said front surface;
a rear surface;
a tab and a fastener positioned on said rear surface;
an inner surface;
a pad positioned on said inner surface; and
a testing strip impregnated with a chemical; and
a matrix card.

11. The vitamin testing diaper device of claim 10, wherein the chemical reacts when exposed to a urine and causes a change in a color of the testing strip after the reaction occurs.

12. The vitamin testing diaper device of claim 11, wherein the change of the color of the testing strip indicates the presence of a substance.

13. The vitamin testing diaper device of claim 12, wherein the substance is at least one of a Vitamin K, a Vitamin D, a Vitamin B12, an Iron, a Vitamin C, a Vitamin A, a Calcium, a Vitamin E, an acidic, a protein, a sugar, a ketone, a bilirubin, a Leukocyte or a red blood cell substance.

14. The vitamin testing diaper device of claim 13, wherein a shade of the color of the testing strip indicates a concentration of the substance.

15. The vitamin testing diaper device of claim 14, wherein the matrix card is comprised of a plurality of colored squares and an indicia that identifies the concentration of the substance using the color of the testing strip after being changed by the chemical.

16. The vitamin testing diaper device of claim 10, wherein the body is comprised of at least one of a wood pulp, a sodium polyacrylate fiber, a plastic, a tissue paper, a non-woven fabric or a non-permeable film material.

17. The vitamin testing diaper device of claim 16, wherein the body is comprised of a hypoallergenic material.

18. The vitamin testing diaper device of claim 10, wherein the pad is infused with at least one of an aloe vera, a zinc oxide, a calendula, a lanolin or a petroleum.

19. The vitamin testing diaper device of claim 10, wherein the pair of leg openings are lined with an elastic.

20. The vitamin testing diaper device of claim 10, wherein each of the fastener of the tab of the front surface and the fastener of the tab of the rear surface is an adhesive fastener.
